(19) United States
(12) Patent Application Publication
(10) Pub. No.: US 2007/0276358 A1
(43) Pub. Date: Nov. 29, 2007

(54) TEMPLATE GRID SYSTEM

(75) Inventors: Winston E. Barzell, Sarasota, FL (US); Willet F. Whitmore, Sarasota, FL (US); Stephen E. Brauner, Bradenton, FL (US); Roger Wilson, Sarasota, FL (US)

Correspondence Address: STEPTOE & JOHNSON LLP, 1330 CONNECTICUT AVENUE, N.W., WASHINGTON, DC, 20036, US

(73) Assignee: CIVCO Medical Instruments, Inc.

(21) Appl. No.: 11/739,091

(22) Filed: Apr. 23, 2007

Related U.S. Application Data
(63) Continuation of application No. 10/636,457, filed on Aug. 7, 2003, now abandoned.
(60) Provisional application No. 60/401,829, filed on Aug. 7, 2002.
(60) Provisional application No. 60/450,785, filed on Feb. 28, 2003.

(51) Int. Cl. A61B 17/00

(52) U.S. Cl. 606/1

(57) ABSTRACT

A template grid is provided for use in positioning medical implants. The template grid includes apertures defining passages extending through the template grid. The passages are sized to accommodate a medical implant such as needles. The template grid further includes an elongated slot. The elongated slot allows a needle to be placed in Denonvilliers space at a desired angle so that the injection of fluid can be accomplished. The elongated slot also allows at least one posteriorly angled treatment probe to be placed below the urethra into the prostate to treat otherwise difficult to reach areas. The template grid can be made in multiple components so that it can be used in different configurations to suit the user's needs.

### CROSS-REFERENCE TO RELATED APPLICATIONS

This application claims the benefit under 35 U.S.C § 119 (e) of U.S. Provisional Patent Application No. 60/401,829, filed Aug. 7, 2002 and entitled TEMPLATE GRID SYSTEM, and to U.S. Provisional Patent Application No. 60/450,785, filed Feb. 28, 2003 and also entitled TEMPLATE GRID SYSTEM, the entirety of which are incorporated herein by reference.

### FIELD OF THE INVENTION

This invention relates generally to a template grid system for positioning medical implants, and more particularly to a template grid system for use with transrectal ultrasound imaging probes for cancerous prostate and related surgeries. The present invention also relates to an anchor for fixing a thermocouple probe with respect to the template grid system.

### BACKGROUND OF THE INVENTION

Brachytherapy (radioactive seeds), thermotherapy (heating), and cryotherapy (freezing) are proven therapies for tumors, both benign and malignant. Although the effectiveness of these treatments has been established, the risks associated with these treatments prevent or at least inhibit the wide application that they night otherwise achieve. The risks in each instance are related to the difficulties in achieving full control and accurate monitoring of the treatment. The risks of damaging surrounding tissues are present in every case, potentially catastrophic and require great care and experience to control.

In this regard, these therapies are frequently performed in conjunction with an ultrasound imaging probe placed in the rectum to monitor treatment. Although there are many variations of technique for placement of the treatment needles into the prostate under transrectal ultrasound guidance, in essence these can be categorized into two basic approaches. First is the “freehand” technique in which an experienced practitioner makes an estimation of a good entry point on the perineal skin and with ultrasound visualization, guides the treatment needles into their ideal location. The advantage of this approach is the flexibility to angle the needles so that they align with the contour of the prostate.

The second approach is the “direct access” approach which is done using a template grid. The template grid arrangement, which is kept in precise linear orientation with the ultrasound probe, must be accurately oriented adjacent the perineum in relation to the prostate, and locked in position throughout the procedure to achieve optimum results. This technique is easier for the novice because it provides a guide to the perineal entry points and a predictable endpoint for the treatment needles. A disadvantage of this approach is that all of the treatment needles are fixed in a parallel relationship to the transrectal ultrasound probe and to each other, and following the prostate contour to more easily create the ideal treatment field is not possible.

One commonly available template grid used to guide placement of the needles is a block of plastic or metal with multiple machined parallel channels arranged in a matrix and spaced at 5 mm intervals. Although these template grids do allow for controlled placement of the treatment needle, there is no provision for ancillary monitoring or treatment instrumentation. For example, it would be advantageous to have a mechanism in which one or more temperature probes, such as thermocouples, could be placed in the tissue being treated during cryotherapy or thermotherapy. With prior art template grids, the template grid must be removed or, the temperature probes, which are typically much smaller in diameter that the machined parallel holes of the grid, can only be placed in one of the machined parallel channels. Given the relative diameters, the temperature probes are unstable in the template grid.

Furthermore and as previously noted, because the posterior surface of the prostate and the anterior wall of the rectum are very close anatomically, treatment (such as freezing or heating) of the prostate risks injury to the rectum. One method for dealing with this possible complication is to inject fluid, such as saline, into Denonvilliers space, the potential space between two layers of fascia that lay between the rectum and the prostate. To approach and enter this space reliably with a needle, it must be done transperineal at a steep angle to the normal alignment of the parallel channels in the template grid. Also, the fluid-containing needle must be placed centrally, so that the template grid obstructs the desired position.

As the previous discussion illustrates, a need for an improved template grid system exists.

### SUMMARY OF THE INVENTION

The present invention discloses a template grid for use in positioning a medical implant. The template grid includes a plurality of apertures defining a plurality of passages extending through the template grid. The passages are sized to accommodate a medical implant such as needles. In one embodiment, the template grid further includes an elongated slot. The elongated slot allows a needle to be placed in Denonvilliers space at a desired angle so that the injection of fluid can be accomplished. The elongated slot also allows at least one posteriorly angled treatment probe to be placed below the urethra into the prostate to treat otherwise difficult to reach areas.

In other embodiments the template grid is made of multiple components so that the template grid can be used in different configuration to suit the user's needs.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front view of an embodiment of the template grid system according to the present invention;

FIG. 2 is a cross-sectional view taken along line A—A of FIG. 1;

FIG. 3 is a front view of an embodiment of the template grid system according to the present invention;

FIG. 4 is an exploded side vide of an embodiment of the template grid system according to the present invention;

FIG. 5 is a perspective view of a thermocouple anchor according to the present invention;

FIG. 6 is a perspective view of the thermocouple anchor of FIG. 5 in use with the template grid of FIG. 1 or 3;

FIG. 7 is another embodiment of a thermocouple anchor according to the present invention:

FIG. 8 is a side view of another embodiment of a template grid system according to the present invention;

FIG. 9 is an enlarged view of the circled portion shown in FIG. 8;

FIG. 10 is a front view of two possible proximal plates that can be removably used with the template grid of FIG. 8;

FIG. 11 is a perspective view of another embodiment of a template grid system according to the present invention;

FIG. 12 is a perspective view of one member of the template grid system of FIG. 9;

FIG. 13 is a perspective view of another embodiment of a template grid system according to the present invention; and

FIG. 14 is an exploded view of the template grid system of FIG. 13.

### DETAILED DESCRIPTION OF THE INVENTION

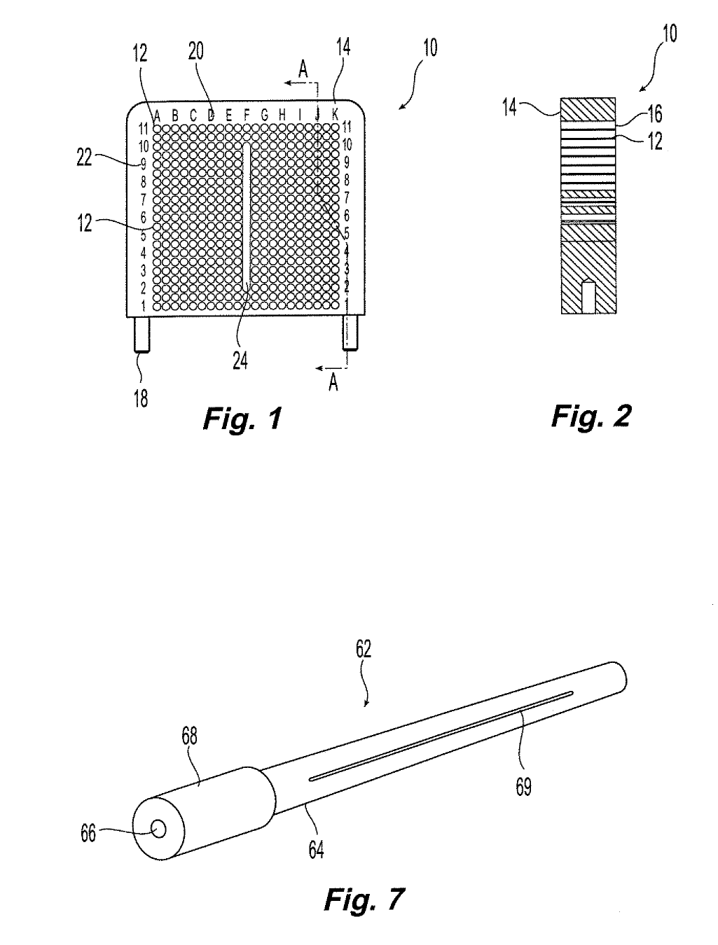

Referring now to the drawing figures in which like reference designators refer to like elements, there is shown in FIGS. 1 and 2 a template grid 10 according to the present invention. Template grid 10 has a series of passages 12 extending from a front 14 through a back 16. Passages 12 are sized to accommodate a medical implant such as needles and can be chamfered at front 14 and/or back 16 to facilitate insertion of the implant. The conical shape of the chamfer enables the implant to be easily inserted into the channels. Passages 12 are arranged in a pattern corresponding to a software-generated pattern superimposed on images from an imaging probe. Template grid 10 can be provided with a recess (not shown) for providing clearance for a portion of the probe.

Template grid 10 has at least one mating element for connection to a grid supporting member. Generally, at least two mating elements are used. Preferably, a pair of prongs 18 operatively associated with a pair of grooves on the grid supporting member, are used for this connection. A preferred arrangement is shown, but the number and location of prongs 18 can be varied as desired to suit the configuration of the grid supporting member.

In order to help the user distinguish the passages 12 from each other, front 14 and/or back 16 have letters 20 identifying the various columns of passages 12 and numbers 22 identifying the various rows of passages 12. As users have different preferences for the manner in which passages 12 are identified, the configuration of the letters 20 and numbers 22 on front 14 need not be identical to the configuration on back 16. Letters 20 and numbers 22 can be made on front 14 and back 16 by a number of methods including silk screening, molding, engraving, or chemical etching. Any other method which provides a permanent placement of these indicia on the template can be used.

Template grid 10 is provided with a slot 24 that has a length substantially larger than its width. Slot 24 extends from front 14 through back 16. As shown, slot 24 is centrally located on template grid 10 and runs vertically. Thus, slot 24 is placed in a position that replaces a column of normally unused passages. However, slot 24 can be made to run horizontal or at any desired orientation. Additionally, slot 24 can be placed anywhere on template grid 10.

Slot 24 allows a needle to be placed in Denonvilliers space at a desired angle so that the injection of fluid can be accomplished. Slot 24 also allows at least one posteriorly angled treatment probe to be placed below the urethra into the prostate to treat otherwise difficult to reach areas.

Template grid 10 can be made of any suitable material. Template grid 10 can be made to be reusable or disposable. In this regard, U.S. Pat. No. 6,036,632, the contents of which are incorporated herein by reference, discloses disposable template grid systems. Template grid 10 can be sterilized by steam sterilization, gas sterilization, or irradiation.

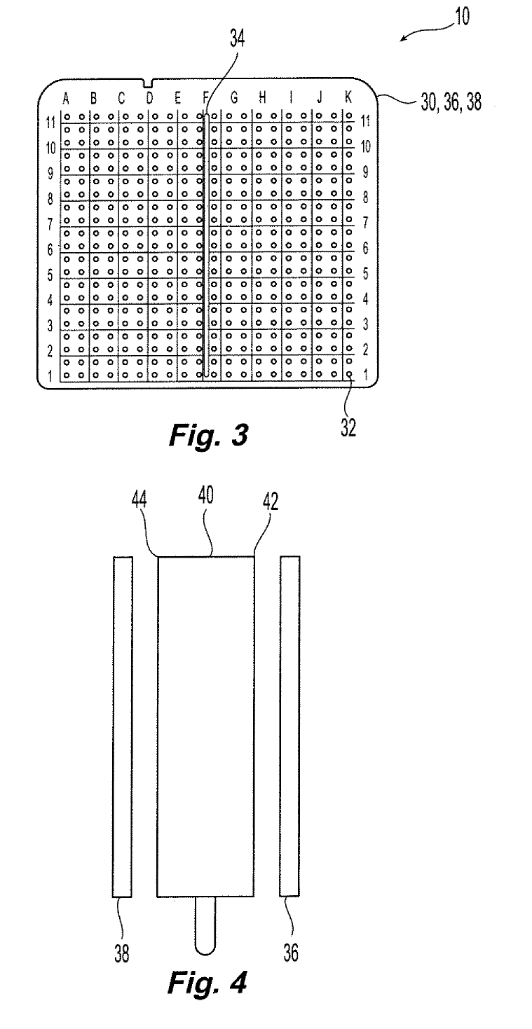

Referring to FIG. 3, the template grid 10 includes at least one element 30 including a plurality of apertures 32. The apertures 32 define a plurality of passages each of which is configured for receiving a medical implant such as needles and can be chamfered to facilitate insertion of the implant. The conical shape of the chamfer enables the implant to be easily inserted into the channels. The passages are arranged in a pattern corresponding to a software-generated pattern superimposed on images from an imaging probe.

The at least one element 30 further includes an elongated slot 34 extending through the at least one element 30, and has a length substantially larger than its width. As shown, the elongated slot 34 is centrally located on template grid 10 and runs vertically. Thus, the slot 34 is placed in a position that replaces a column of normally unused passages. However, the slot 34 can be made to run horizontal or at any desired orientation. Additionally, the slot 34 can be placed anywhere on template grid.

In accordance with the present invention, as shown in FIGS. 3 and 4, the at least one element 30 includes a first element 36 having a plurality of apertures 32 and elongated slot 34 and a second element 38 including a plurality of apertures 32 and elongated slot 34. The first and the second elements 36 and 38 align such that the apertures 32 are aligned defining the plurality of passages 32 and the elongated slots 34 are aligned therethrough.

The present invention can further include a support frame 40. The first element 36 is removable attachable to a first side 42 of the support frame 40 and the second element 38 is removable attachable to a second side 44 of the support frame 40. The first element 36 and the second element 38 are positioned on the support frame 40 such that apertures 32 are aligned defining the plurality of passages therethrough and the elongated slots 34 are aligned theretirough.

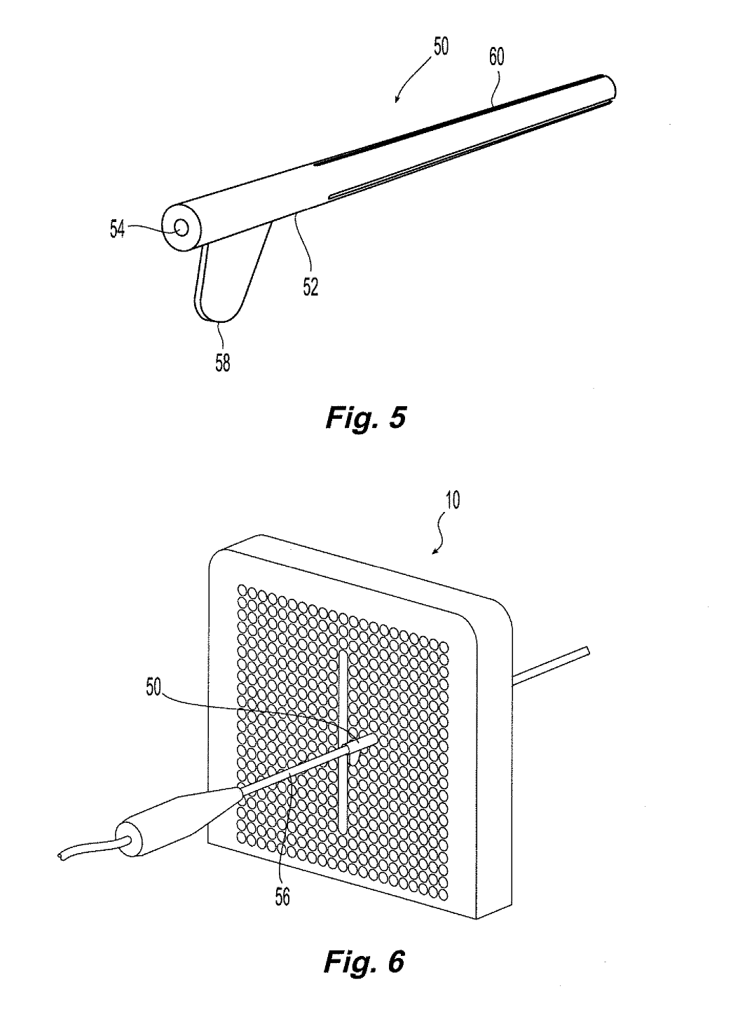

FIG. 5 shows a thermocouple probe anchor 50 according to the present invention. Thermocouple probe anchor 50 has a tapered body 52 and is provided with a lumen 54 that extends through body 52. As best seen in FIG. 6, lumen 54 is configured and dimensioned to receive a thermocouple probe 56 and body 52 is configured and dimensioned to fit in any one of passages 12. As a result, thermocouple probe 56 is securely positioned with respect to template grid 10. Body 52 includes a tab 58 that facilitates handling and manipulation of anchor 50. Additionally, tab 58 can be positioned so that tab 58 limits the extent anchor 50 can be inserted in passage 12. Alternatively, this can be achieved with the shape and size of the taper of body 52. Body 52 is provided with one or more grooves 60 that allow contraction and expansion of body 52.

FIG. 7 shows another embodiment of a thermocouple probe anchor 62 according to the present invention. In general, most of the structure shown in FIG. 7 is like or comparable to the structure illustrated in the embodiment shown in FIGS. 5 and 6 and accordingly discussion of those like components is not believed necessary. Thermocouple probe anchor 62 has a tapered body 64 and is provided with a lumen 66 that extends through body 64. Lumen 66 is configured and dimensioned to receive a thermocouple probe and body 64 is configured and dimensioned to fit in any one of passages in a template grid (such as passages 12 of template grid 10 in FIG. 1). As a result, the thermocouple probe is securely positioned with respect to the template grid 10. Body 64 includes a head 68 that facilitates handling and manipulation of anchor 62. Additionally, head 68 can be positioned so that head 68 limits the extent anchor 62 can be inserted in passage 12. Alternatively, this can be achieved with the shape and size of the taper of body 64. Body 64 is provided with one or more grooves 69 that allow contraction and expansion of body 64. Unlike grooves 60, grooves 69 do not extend to the distal end of body 64. However, the lengths of grooves 60 and 69 can be varied to help achieve proper fitting of the thermocouple probes and secure insertion into passages 12.

Like template grid 10, anchors 50 and 62 can be made of any suitable material and can be made to be reusable or disposable. Anchors 50 and 62 can be sterilized by steam sterilization, gas sterilization, or irradiation.

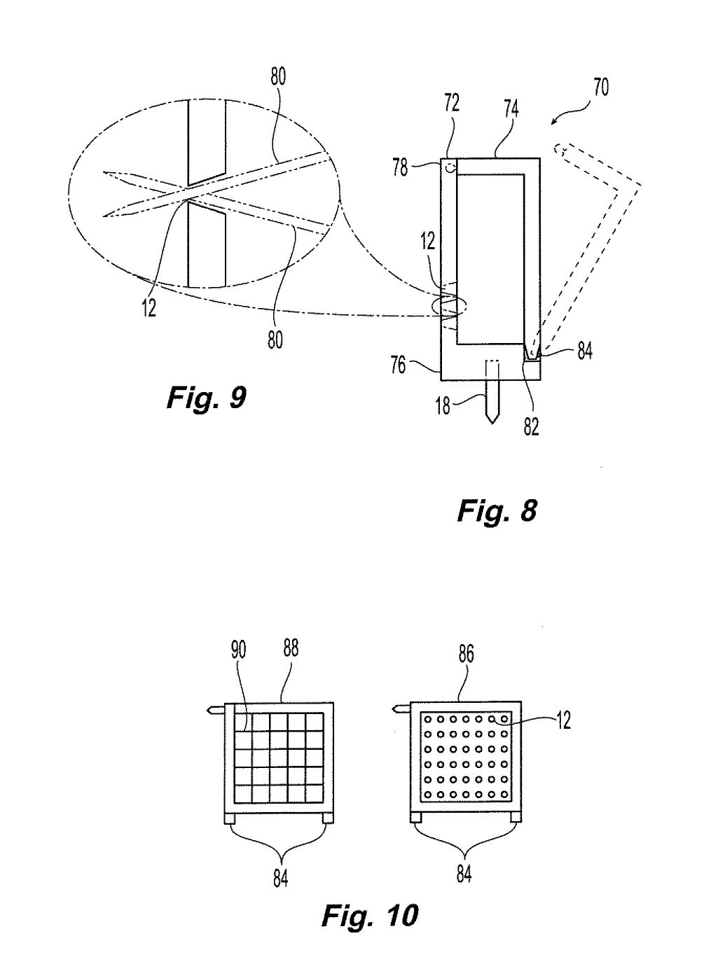

FIG. 8 shows another embodiment of a template grid 70 according to the present invention. In general, most of the structure shown in FIG. 8 is like or comparable to the structure illustrated in the embodiment shown in FIGS. 1-4 and accordingly discussion of those like components is not believed necessary. Template grid 70 includes an L-shaped distal member 72 and an L-shaped proximal member 74. The terms “proximal” and “distal” are with respect to the surgeon or user (as opposed to the patient) and are used only for convenience. Distal member 72 includes a base 76 and a distal plate 78. Base 76 can be provided with prongs 18 for connection to a grid supporting member. Alternatively, other connecting mechanisms can be used.

Distal plate 78 has a series of passages 12 sized to accommodate a medical implant such as needles. As best seen in FIG. 9, passages 12 are configured and dimensioned so that needle 80 can be inserted at an angle of about 30° off perpendicular in any direction. There are a number of different ways of making passages 12 this way. For example, passages can be chamfered or oversized. Alternatively, distal plate 78 could be made thin enough to deform and allow angulation of needles 80. In another embodiment, an elastic member could be placed in the passages 12 (for example lining the wall of passage 12) to provide for angular insertion of needles 80.

Angulation of needles 80 is possible only when proximal member 74 is removed or proximal member 74 is configured to allow angulation. In particular, distal member 72 and proximal member 74 are each provided with recesses 82 and/or snap-in fasteners 84 so that proximal member 74 can be removably coupled to distal member 72. Other known mechanisms of removably connecting two components can be used. FIG. 10 shows two possible proximal members that can be used. A fixed plate 86 contains passages 12 similar to the passages on distal plate 78. Because the passages on fixed plate 86 are substantially parallel to the passages on distal plate 78, little or no angulation of needles 80 is possible. A slotted plate 88 is made of a grid of wires 90 or other filaments. Wires 90 provide some guidance, yet still allows angulation of needles 80.

In summary, template grid 70 has three separate configurations. If proximal member 74 is removed, free angulation of needles 80 is possible. This is likely to be the configuration preferred by experienced users as it gives the most flexibility for needle placement. In contrast, the use of fixed plate 86 is likely to be preferred by the inexperienced user as this configuration provides the most guidance in terms of needle placement. The use of slotted plate 88, the third configuration, is a hybrid that provides some guidance, yet still provides angulation of needles.

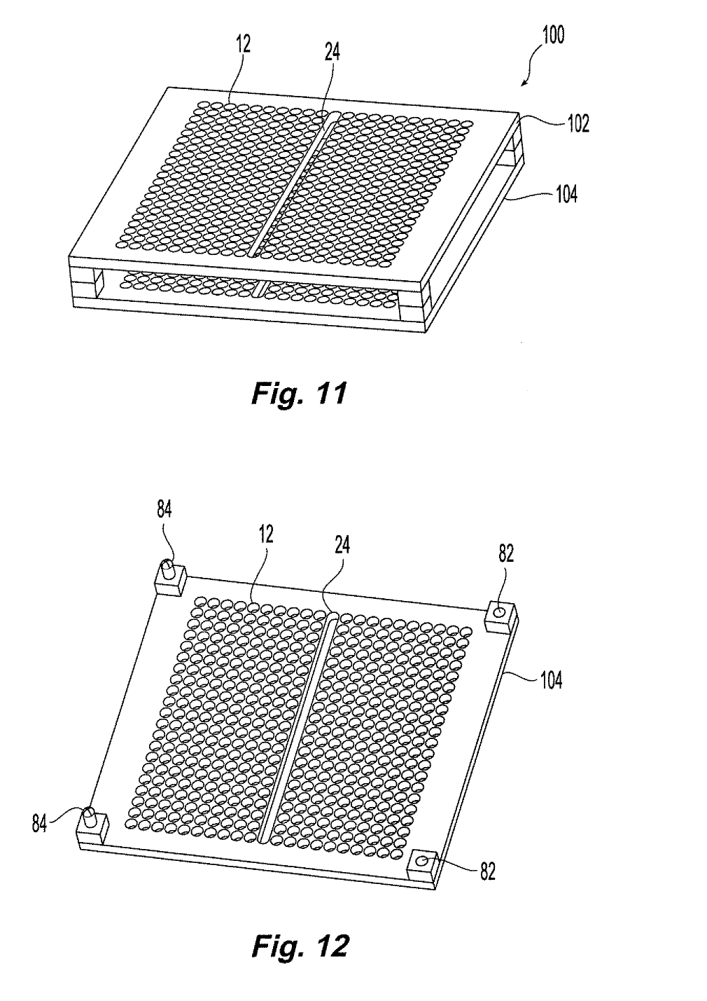

FIGS. 11 and 12 show another embodiment of a template grid 100 according to the present invention. In general, most of the structure shown in FIGS. 11 and 12 is like or comparable to the structure illustrated in the embodiment shown in FIG. 6 and accordingly discussion of those like components is not believed necessary. Specifically, template grid 100 has proximal and distal members 102, 104 that are removably coupled. Passages 12 in distal member 104 are configured and dimensioned so that when proximal member 102 is not attached, angled placement of the needles is possible. In contrast, when proximal and distal members 102, 104 are coupled, parallel placement of needles is achieved.

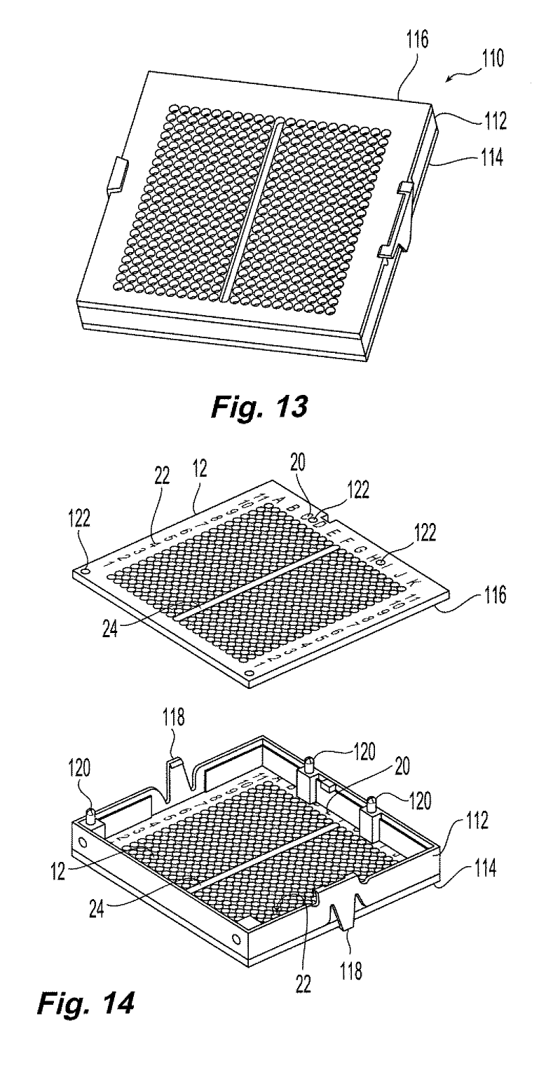

FIGS. 13 and 14 show generally a template grid 110 according to the present invention. In general, most of the structure of template grid 110 is like or comparable to the structure of the other template grids according to the present invention and accordingly discussion of those like components is not believed necessary. Template grid 110 includes a frame 112 to which distal and proximal plates 114 and 116 are removably coupled. Although it is not necessary to have distal plate 116 removable from frame 112, it has been found useful for manufacturing ease as well as other practical considerations. For example, as optimized surgical techniques develop, being able to adapt both distal and proximal plates 114 and 116 could be beneficial. Frame 112 has resilient tabs 118 so that plates 114 and 116 can be easily removed. Frame 112 and distal and proximal plates 114 and 116 are provided with alignment pins 120 and bores 122 to help ensure proper placement of the components.

Distal and proximal plates 114 and 116 are provided with a series of passages 12 sized to accommodate a medical implant such as needles. As was the case with the passages for the other template grids, passages 12 are configured and dimensioned to allow angulation of the needles. Thus, when proximal plate 116 is removed from frame 112, full angulation of the needles is possible. When proximal plate 116 is connected to frame 112, the passages on proximal plate 116 line up with the passages on distal plate 114. This permits only parallel placement of the needles. Distal and proximal plates 114 and 116 are provided with indicia (such as numbers and/or letters) identifying passages 12. These indicia can be located on both the front and back of plates 114 and 116. As a result, the indicia are viewable regardless of whether proximal plate 116 is used.

While various embodiments of the present invention are described above, it should be understood that the various features could be used singly or in any combination thereof. Therefore, this invention is not to be limited to only the specifically preferred embodiments depicted herein.

Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. A template grid comprising:
a plurality of apertures defining a plurality of passages each of which is adapted to receive a first medical device therethrough; and
an elongated slot adapted to receive a second medical device therethrough.

2. The template grid according to claim 1, further comprising a first element including a plurality of first element apertures and a first element elongated slot.

3. The template grid according to claim 2, wherein the first element includes a plurality of markers to identify each of the plurality of first element apertures.

4. The template grid according to claim 2, wherein each of the plurality of first element apertures is chamfered.

5. The template grid according to claim 2, further comprising a second element including a plurality of second element apertures and a second element elongated slot, wherein the first element apertures and the second element apertures are aligned defining the plurality of passages.

6. The template grid according to claim 5, wherein the second element includes a plurality of markers to identify each of the plurality of second element apertures.

7. The template grid according to claim 5, wherein each of the plurality of second element apertures is chamfered.

8. The template grid according to claim 5, wherein the first element elongated slot and the second element elongated slot are aligned defining the elongated slot.

9. The template grid according to claim 1, further comprising a slotted plate including filament members defining a grid, wherein the grid is substantially aligned with the plurality of passages.

10. The template grid according to claim 1, further comprising at least one element including the plurality of apertures defining the plurality of passages and an elongated aperture defining the elongated slot.

11. The template grid according to claim 10, further comprising a support fame configured for receiving the at least one element.

12. The template grid according to claim 11, wherein the at least one element includes a first element having a plurality of first element apertures and a first element elongated slot, the first element being attachable to a first side of the support frame.

13. The template grid according to claim 12, further comprising a second element including a plurality of second element apertures and a second element elongated slot, the second element being attachable to a second side of the support frame, wherein the first element apertures and the second element apertures are aligned defining the plurality of passages and the first element elongated slot and the second element elongated slot are aligned defining the elongated slot.

14. The template grid according to claim 1, wherein the elongated slot is centrally located on the template grid.

15. The template grid according to claim 1, wherein the elongated slot is vertically orientated on the template grid.

16. The template grid according to claim 1, wherein the elongated slot is horizontally orientated on the template grid.

17. The template grid according to claim 1, further comprising at least one prong for connection to a grid support member.

18. A template grid comprising:
at least one element including a plurality of apertures defining a plurality of passages and an elongated slot, each of the plurality of passages being adapted to receive a first medical device and the elongated slot being adapted to receive a second medical device therethrough; and
a support frame configured for receiving the at least one element.

19. The template grid according to claim 18, wherein the at least one element includes a plurality of markers to identify each of the plurality of passages.

20. The template grid according to claim 18, wherein the each of the plurality of apertures is chamfered.

21. A template grid comprising:
a proximal plate having a plurality of proximal channels each of which is adapted to receive a medical implant;
a distal plate having a plurality of distal channels each of which is adapted to receive the medical implant; and
a support frame having proximal and distal sides, the proximal plate removably attached to the proximal side and the distal plate attached to the distal side such that each of the proximal channels aligns with a corresponding distal channel thereby defining a plurality of passages adapted to allow the medical implant to pass from and through the proximal plate to and through the distal plate when the proximal and distal plates are attached to the support frame,
wherein each of the distal channels is configured and dimensioned to allow angulation of the medical implant when the proximal plate is removed from the support frame.

22. The template grid of claim 21 wherein:
the proximal plate includes a proximal slot;
the distal plate includes a distal slot; and
the proximal and distal slots align when the proximal and distal plates are attached to the support frame thereby defining an elongated slot having a length substantially larger than a width.
